(19) United States
(12) Patent Application Publication
(10) Pub. No.: US 2013/0338682 A1
(43) Pub. Date: Dec. 19, 2013

(54) Tissue Ligation Devices and Methods

(71) Applicant: COOK MEDICAL TECHNOLOGIES LLC, Bloomington, IN (US)

(72) Inventor: Jeffry S. Melsheimer, Springville, IN (US)

(21) Appl. No.: 13/918,349

(22) Filed: Jun. 14, 2013

Related U.S. Application Data
(60) Provisional application No. 61/660,089, filed on Jun. 15, 2012.

(51) Int. Cl. A61B 17/12

(52) U.S. Cl. CPC A61B 17/12009
USPC 606/140

(57) ABSTRACT

Medical devices useful in the ligation of tissue, apparatuses useful in the making and using of the medical devices, and methods of making and using the medical devices are described. A method of ligating tissue includes placing a ligating band on an introducer, stretching the ligating band, transporting the ligating band to a position adjacent a tissue using the introducer, passing the ligating band over the tissue, and releasing the ligating band from the introducer to place the ligating band in an unstretched configuration around the tissue. A ligating band includes an elongate fiber having first and second ends and forming a series of loops. The second end can be free of direct securement to the first end and secured to a portion of the fiber positioned within a loop of the series of loops.

### FIELD

The invention relates to medical devices. More particularly, the invention relates to medical devices useful in the ligation of tissue, apparatuses useful in the making and using of the medical devices, and methods of making and using the medical devices.

### BACKGROUND

It is sometimes necessary or otherwise desirable to ligate a tissue or portion of a tissue in an animal. Ligations can be performed to block entry to a cavity, to restrict the flow of nutrients to a tissue or portion of a tissue, or to remove a tissue or portion of a tissue from a body.

For example, the left atrial appendage (LAA), a relatively small, thumb-shaped cavity located off the left atrium between the mitral valve and the left pulmonary vein, defines a blind sac-like chamber. In some individuals, the LAA may not contract with enough force to completely expel blood from the chamber as the heart contracts. Stagnant blood can collect in the LAA as a result, which may increase the potential for the formation of blood clots and/or thrombi and the susceptibility to stroke and other clinical conditions.

Several tissues, including the LAA, can be removed from the body without causing any known issues. Some of these tissues are challenging to remove by surgical procedures, though. The irregular shape, volume, and texture of the LAA lining, for example, make its surgical removal difficult.

There is a need, therefore, for improved devices and methods that can be used in the ligation of tissues or portions of tissues, such as the LAA.

### BRIEF SUMMARY OF THE DISCLOSURE

Methods of ligating tissue are described.

An exemplary method comprises stretching a ligating band that defines a loop to place the ligating band in a stretched configuration, transporting the ligating band to a position in the body adjacent the tissue to be ligated, passing the ligating band over the tissue to be ligated while the ligating band is in its stretched configuration, and returning the ligating band to its unstretched configuration on the tissue to be ligated so that the ligating band surrounds and constricts the tissue. The method can optionally include a step of inducing an inflammatory response in the tissue, such as at one or more points of contact between the ligating band and the tissue to be ligated.

Another exemplary method comprises transporting a ligating band to a position in the body adjacent the LAA of an animal, stretching the ligating band by applying a pulling force to the ligating band, passing the ligating band over the LAA, and removing the pulling force from the ligating band so that the ligating band surrounds and constricts the LAA. The method can optionally include a step of inducing an inflammatory response in the tissue, such as by applying an electrical current to the exterior lining of the LAA at one or more points of contact between the ligating band and the LAA.

Ligating bands are also described.

An exemplary ligating band comprises a series of individual loops formed in a continuous silk fiber, such as a fiber of spider silk.

Methods of making ligating bands are also described.

An exemplary method comprises securing a first end of an elongate fiber to a spool, wrapping the elongate fiber around the spool to form a loop with the elongate fiber, repeating the looping step a suitable number of times to form a series of loops with the elongate fiber, adhering a second end of the elongate fiber to a portion of the elongate fiber positioned in an existing loop on the spool, and transferring the ligating band onto an introducer.

Introducers for holding and transporting a ligating band to a position adjacent a tissue to be ligated are also described.

An exemplary introducer comprises an elongate tubular member, first and second hollow and extendable arms disposed through the passageway of the elongate tubular member, and a separating bridge disposed in the distal end of the passageway of the elongate tubular member. Each of the arms is biased radially outward, and has an engaging member disposed within its inner passageway. The engaging members can be looped around a ligating band disposed between the arms such that the ligating band is stretched when the arms are advanced distally.

### BRIEF DESCRIPTION OF THE DRAWINGS

FIG. 1 is a flowchart representation of an exemplary method of ligating tissue.

FIG. 2 is a perspective view of an exemplary ligating band.

FIG. 3 is a flowchart representation of an exemplary method of making a ligating band.

FIG. 4 is a perspective view of a spool useful for holding and shaping a ligating band during its making and that facilitates transfer of the ligating band to an introducer.

FIG. 5 is a partial side view of an introducer useful in methods of ligating tissue.

FIG. 5A is a top view of the introducer illustrated in FIG. 5.

FIG. 5B is a partial sectional view of the introducer illustrated in FIG. 5, taken along line 5B-5B in FIG. 5A.

FIG. 5C is a partial perspective view of the introducer illustrated in FIG. 5.

FIG. 5D is a partial side view of the introducer illustrated in FIG. 5.

FIG. 6 is a partial side view of an arm of the introducer illustrated in FIG. 5.

FIG. 6A is a sectional view of the arm illustrated in FIG. 6, taken along line 6A-6A in FIG. 6.

FIG. 7A is a partial perspective view of an introducer with an associated ligating band.

FIG. 7B is a partial perspective view of the introducer illustrated in FIG. 7A. The introducer arms are in extended positions and the ligating band is in a stretched configuration.

FIG. 8 is a partial perspective view of another introducer with an associated ligating band.

FIG. 8A is a sectional view of the introducer illustrated in FIG. 8, taken along line 8A-8A in FIG. 8.

FIG. 9 is a schematic of a patient chest cavity in which the introducer illustrated in FIG. 8 is being used in a method of ligating tissue.

FIG. 10A is a schematic of a patient chest cavity in which the introducer illustrated in FIG. 8 is positioned for one step in a method of ligating tissue.

FIG. 10B is a schematic of a patient chest cavity in which the introducer illustrated in FIG. 8 is positioned for another step in a method of ligating tissue.

FIG. 10C is a schematic of a patient chest cavity in which the introducer illustrated in FIG. 8 is positioned for another step in a method of ligating tissue.

FIG. 10D is a schematic of a patient chest cavity in which the introducer illustrated in FIG. 8 is positioned for another step in a method of ligating tissue.

FIG. 10E is a schematic of a patient chest cavity in which the introducer illustrated in FIG. 8 is positioned for another step in a method of ligating tissue.

FIG. 10F is a schematic of a patient chest cavity in which the introducer illustrated in FIG. 8 is positioned for another step in a method of ligating tissue.

### DETAILED DESCRIPTION

The following detailed description and the appended drawings are provided to describe and illustrate exemplary embodiments of the invention for the purpose of enabling one of ordinary skill in the relevant art to make and use the invention. The description and drawings are not intended to limit the scope of the invention or its protection in any manner.

As used herein, the term “tissue” refers to biological tissue and tissue portions, such as animal tissue, including human tissue. The term does not refer to biological organs in their entirety, and encompasses entire organs, portions of organs, and other biological tissues.

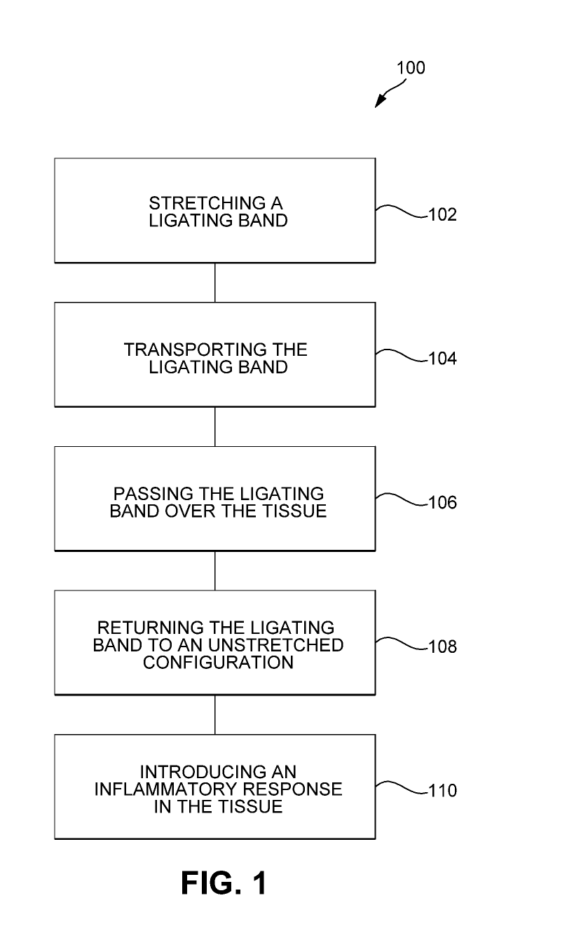

FIG. 1 illustrates an exemplary method 100 of ligating tissue.

An initial step 102 comprises stretching a ligating band that defines a loop to place the ligating band in a stretched configuration. This step results in the ligating band having an inner diameter that is larger than a maximum outer diameter of the tissue to be ligated. Advantageously, the inner diameter of the loop is smaller than the maximum outer diameter of the tissue when the ligating band is in a resting or unstretched configuration. This ensures that the ligating band will constrict the tissue once secured around it.

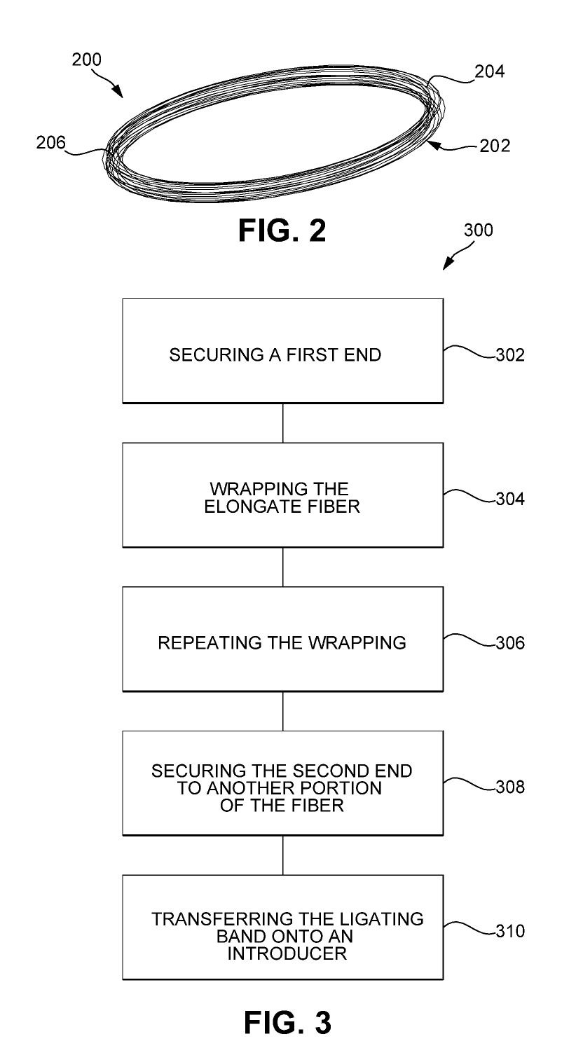

Any suitable ligating band can be used in the method 100, and a skilled artisan will be able to select an appropriate ligating band for a particular method based on various considerations, including the size, configuration and location of the tissue to be ligated. The ligating band need only be capable of performing as described in the method. Thus, the ligating band should be capable of being stretched to a degree that provides stretched and unstretched configurations of suitable size for the tissue to be ligated, of being transported to the tissue to be ligated, of being passed over the tissue to be ligated while in a stretched configuration, and of constricting the tissue to be ligated once returned to an unstretched configuration after being passed over the tissue. Also, the ligating band should be biocompatible or capable of being rendered biocompatible. The ligating band 200 illustrated in FIG. 2 and described below is considered a suitable ligating band for use in the methods of ligating tissue contemplated by the inventor.

This step 102 can be conducted using any suitable technique for stretching a band, and a skilled artisan will be able to select an appropriate technique for a particular method based on various considerations, including the size of the ligating band, the elasticity of the ligating band, and other considerations. Examples of suitable techniques for stretching a band include applying an outwardly-directed force onto two or more positions on the ligating band from points external to the ligating band (i.e., applying a pulling force onto two or more positions on the ligating band), and applying an outwardly-directed force onto two or more positions on the ligating band from points internal to the ligating band (i.e., applying a pushing force onto two or more positions on the ligating band).

Another step 104 comprises transporting the ligating band to a position in the body adjacent the tissue to be ligated. This step can be accomplished using suitable techniques and devices, and a skilled artisan will be able to select appropriate techniques and devices based on various considerations, including the location of the tissue in the body of the animal being treated. For example, in a method of ligating the LAA of a patient, the ligating band can be transported to the location of the LAA using an introducer, such as the introducer described in detail below, to gain access through the chest/abdominal wall.

The step 104 of transporting the ligating band to a position adjacent the tissue to be ligated can be performed before, simultaneously with, or after the step 102 of stretching the ligating band to an inner diameter that is larger than a maximum outer diameter of the tissue to be ligated. For example, the ligating band can be stretched while external to the patient, and then transported to a position adjacent the tissue to be ligated using a device that holds the ligating band in its stretched configuration during transport. Alternatively, the ligating band can be loaded onto a device and held in its resting configuration during transport. Once at the position adjacent the tissue to be ligated, the ligating band can then be stretched. A skilled artisan will be able to select an order of these steps for a particular method of ligating tissue based on various considerations, including the location of the tissue to be ligated within the body, the ability to maintain the ligating band in a stretched configuration during transport, and other considerations.

Another step 106 comprises passing the ligating band over the tissue to be ligated while the ligating band is in its stretched configuration. This step can be conducted using any suitable technique, and a skilled artisan will be able to select an appropriate technique and approach for a particular method based on various considerations, including the size and configuration of the tissue to be ligated. For example, if the position of the tissue to be ligated is relatively fixed in the body, i.e., the tissue does not move a significant amount when pushed or otherwise contacted, the ligating band can be passed over the tissue by simply advancing the transportation device toward the tissue such that the tissue passes through the central aperture defined by the ligating band. The inventor has determined that this technique is suitable for methods of ligating the LAA. Alternatively, if the tissue moves or may move in response to contact, a force can be applied to an opposing surface or position on the tissue, such as with a finger or another device, while the ligating band is advanced over the tissue by advancing the transportation device toward the tissue from the opposite side of the tissue such that the tissue passes through the central aperture defined by the ligating band.

Another step 108 comprises returning the ligating band to its unstretched configuration on the tissue to be ligated so that the ligating band surrounds and constricts the tissue. This step 108 can be conducted using any suitable technique, and a skilled artisan will be able to select an appropriate technique for a particular method based on various considerations, including the technique for stretching a band used in step 102 and the technique and/or device for transporting the ligating band used in step 104. For example, if the step 102 of stretching the band was conducted by applying a pulling force onto the band, the pulling force can simply be removed gradually or suddenly so that the band returns to its unstretched configuration.

An optional step 110 comprises inducing an inflammatory response in the tissue. Skilled artisans will be able to determine if the inclusion of this optional step is desired or appropriate in a particular method based on various considerations, including the desire or need for an additional factor contributing to the necrosis of the ligated tissue, and other considerations. The inventor has determined that the inclusion of this optional step is advantageous in ligations of tissues that define passageways or cavities for which closure is desired. For example, in a ligation of the LAA, the closure of the blind sac-like chamber at the time of placement of the ligating band is desirable as doing so eliminates the ability of the chamber to collect stagnant blood, providing beneficial effects before the necrosed LAA sloughs off as a result of the ligation.

If included, the step 110 of inducing an inflammatory response can be performed at any suitable location in the body relative to the point or points of contact between the ligating band and the tissue to be ligated. A skilled artisan will be able to select an appropriate location for a particular method based on various considerations, including the nature and location of the tissue to be ligated. Examples of suitable locations include at one or more points of contact between the ligating band and the tissue to be ligated, and points on the tissue to be ligated that are free of contact with the ligating band. Also, in ligations of tissues that define passageways or cavities for which closure is desired, the location can be at a point within the passageway or cavity. For example, in a LAA ligation as described herein, an inflammatory response can be induced in the tissue defining the interior surface of the LAA before, during or after placement of the ligating band around the exterior of the LAA.

If included, the step 110 of inducing an inflammatory response can be conducted using any suitable technique, device, and/or material. A skilled artisan will be able to select an appropriate technique, device, and/or material for a particular method based on various considerations, including the nature and location of the tissue to be ligated. For example, if the tissue to be ligated is readily accessible during performance of the method, inflammation can be induced by ablating the tissue by application of an electrical current using conventional tissue ablation techniques and devices. Alternatively, an inflammation-inducing material, substance, or drug can be applied to the outer surface of the tissue. Also alternatively, the tissue can simply be mechanically disrupted to induce an inflammatory response, such as by cutting, tearing or otherwise injuring the tissue using an appropriate device or material, such as forceps, scissors, a needle, and the like. Similarly, an inflammation-inducing material, substance, or drug can be applied to the ligating band such that the material, substance or drug contacts the tissue following placement of the ligating band around the tissue to be ligated.

Once the method 100 is completed, the ligating band is disposed around and constricting the tissue to be ligated. As a result of the ligation, the tissue will likely become necrotic and may eventually detach from adjoining tissue at or near the position at which the ligating band contacts the tissue. Following detachment, the sloughed tissue may be resorbed by the body or, alternatively, may be removed using an interventional procedure, such as a surgical or catheter-based retrieval procedure.

FIG. 2 illustrates an exemplary ligating band 200. The ligating band 200 comprises a continuous loop 202 of material. As illustrated in FIG. 2, the continuous loop 202 can comprise a series 204 of individual loops 206 or windings disposed adjacent one another. Any suitable number of loops 206 can be used to form the series 204 of loops, and a skilled artisan will be able to select an appropriate number of loops for a particular ligating band based on various considerations, including the nature of the material of the ligating band, the desired thickness of the ligating band, and the desired elasticity of the ligating band. Examples of suitable numbers of loops include two to 10 loops, 10 to 100 loops, and 100 to 1000 loops. Alternatively, the continuous loop 202 can comprise a single, endless loop of material.

The ligating band 200 can be formed of any suitable material, including natural and synthetic materials. A skilled artisan will be able to select an appropriate material for a ligating band according to a particular embodiment based on various considerations, including the tissue with which the ligating band is intended to be used, the location of the tissue in the body, and any physical constraints associated with placing the ligating band around the tissue to be ligated. Examples of suitable synthetic materials include latex, such as casein-free latex, nitrile, vinyl, silicone rubber, EPDM, Pebax, and thermoplastic elastomers. Examples of suitable natural materials include natural silk materials, recombinantly produced forms of natural silk materials, and extracellular matrix (ECM) materials, such as small intestine submucosa (SIS) and other bioremodellable materials.

The inventor has determined that natural silks, such a spider silk and silkworm silk, provide suitable materials for ligating band 200. Spider silk is considered particularly suitable at least because it is highly elastic, has relatively high strength, has relatively low weight, sticks to itself when wound into loops, is naturally biocompatible, and is bioabsorbable. Furthermore, spider silk is highly ductile—the material is able to stretch to 140% of its length without breaking and has the ability to supercontract to 50% or less of its original length when exposed to water. These characteristics make spider silk an attractive material for in vivo tissue ligation.

While not as easy to naturally produce and harvest in large scale quantities as silkworm silk, the relatively higher strength, elasticity and bioabsorbability of spider silk currently make it more attractive for the ligating band 200.

While generally considered to be naturally biocompatible, it is noted that spider silk can be treated to remove or reduce the outer sticky layer by exposure to an alkaline solution. Such treatment of the material may be desirable if the ligating band being formed is intended to be used in an individual that is or may be sensitive or hypersensitive to the sticky layer.

Both natural spider silk, such as silk harvested from spiders, and recombinant spider silk are considered suitable materials for ligating bands as described herein. Recombinant spider silk is currently preferred simply because it remains difficult to harvest suitable quantities of natural spider silk, despite recent advancements.

FIG. 3 illustrates an exemplary method 300 of making a ligating band.

An initial step 302 comprises securing a first end of an elongate fiber to a spool.

Another step 304 comprises wrapping the elongate fiber around the spool to form a loop with the elongate fiber.

Another step 306 comprises repeating the wrapping 304 step a suitable number of times to form a series of loops with the elongate fiber. It is considered advantageous to perform this step in a manner that maintains a tension on the elongate fiber that produces 1% or less elongation of the elongate fiber. This repeating step can be performed until any suitable number of loops is produced, and a skilled artisan will be able to select an appropriate number of loops—and a corresponding number of repetitions—for a particular method based on various considerations, including the nature of the material of the ligating band, the desired thickness of the ligating band, and the desired elasticity of the ligating band. Examples of suitable numbers of loops include two to 10 loops, 10 to 100 loops, and 100 to 1000 loops.

Another step 308 comprises securing a second end of the elongate fiber to a portion of the elongate fiber positioned in an existing loop on the spool. The securing can be accomplished in any suitable manner, including by adhering the second end to another portion of the elongate fiber, fastening the second end of the elongate fiber to another portion of the elongate fiber using a suitable structure, such as a clip or other suitable device, and felting the second end of the elongate fiber to another portion of the elongate fiber.

Another step 310 comprises transferring the ligating band onto an introducer. The transferring can be accomplished in any suitable manner, and a skilled artisan will be able to select an appropriate technique based on various considerations, including the nature of the material of the ligating band and the structure of the introducer onto which it is being transferred. An exemplary technique for transferring is described below in connection with the extendible arm loading apparatus.

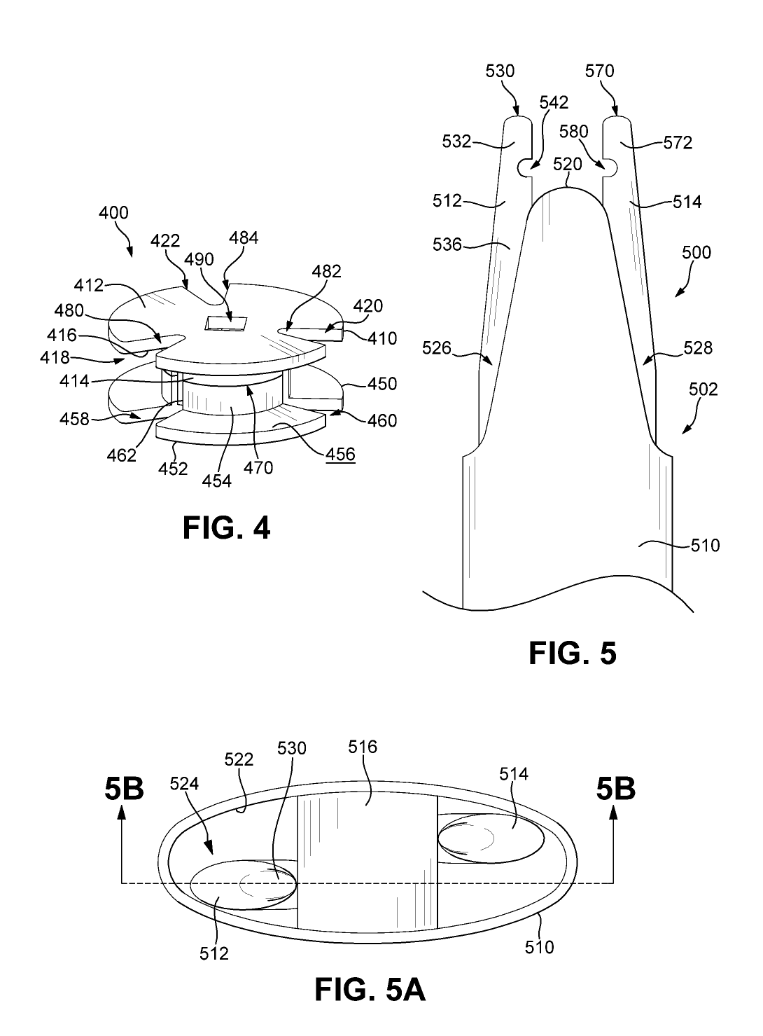

FIG. 4 illustrates a spool 400 for holding and shaping a ligating band during its making. The spool 400 comprises first 410 and second 450 end members that are releasably attached to each other at junction 470. The end members 410, 450 cooperatively define first 480, second 482 and third 484 channels. A central passageway 490 extends through the spool 400.

The first end member 410 includes a flange 412 and a neck portion 414. The neck portion 414 extends away from an inner surface 416 of the end member 410 and has an outer diameter that is less than the outer diameter of the flange 412. The flange 412 defines first 418, second 420, and third 422 notches that extend inward from the outer perimeter of the flange 412. Similarly, the neck portion 414 defines first, second, and third notches (not referenced with numerals in the figure) that extend inward from the outer surface of the neck portion 414.

The second end member 450 has the same construction as the first end member 410. Thus, the second end member 450 includes a flange 452 and a neck portion 454. The neck portion 454 extends away from an inner surface 456 of the end member 450 and has an outer diameter that is less than the outer diameter of the flange 452. The flange 452 defines first 458, second 460, and third (not visible in the figure) notches that extend inward from the outer perimeter of the flange 452. Similarly, the neck portion 454 defines first 462, second (not referenced in the figure), and third (not visible in the figure) notches that extend inward from the outer surface of the neck portion 454.

The first 410 and second 450 end members can be aligned with each other when attached to each other such that the first notch 418 of the first end member 410 is aligned with the first notch 458 of the second end member 450, the second notch 420 of the first end member 450 is aligned with the second notch 460 of the second end member 450, and the third notch 422 of the first end member 410 is aligned with the third notch of the second end member 450. When aligned in this manner, as illustrated in FIG. 2, the notches defined by the neck portions 414, 454 of the end members 410, 450 are also aligned. In this configuration, the notches defined by the flanges 412, 452 and neck portions 414, 545 of the first 410 and second 450 end members cooperatively define the first 480, second 482, and third 484 channels.

Advantageously, each of the notches 418, 420, 422, 458, 460 defined by the flanges 412, 452 extend to the same depth as the notches defined by the neck portions 414, 454 relative to the outer diameter of the neck portions 414, 454. This provides the channels 480, 482 with a constant depth relative to the outer diameter of the neck portions 414, 454, which is expected to enhance transfer a ligating band from the spool 400 to an introducer, as described below.

The first 410 and second 450 end members can include any suitable structure that allows the desired releasable attachment, including mating threads and other mating mechanical connections, opposing magnetic connectors with or without mating mechanical connections, and any other structure suitable for forming a releasable attachment between the first 410 and second 450 end members.

The central passageway 490 can have any suitable configuration. Inclusion of a keyed configuration, such as the illustrated passageway 490 having a substantially square-shaped cross-sectional shape is considered advantageous at least because it allows for placement of the spool onto a mating mandrel that can drive rotation of the spool 400 without slipping or substantially without slipping, which is expected to aid in use of the spool in forming of ligating bands, as discussed below.

The spool 400 can be formed of any suitable material, and a skilled artisan will be able to select a suitable material for a particular spool based on various considerations, including the nature of the material used in ligating bands that will be used with the spool. It is considered advantageous to form the spool 400 of a material to which the material of the ligating band does not adhere readily. For ligating bands formed of silk fibroin, the inventor has determined that various polymeric materials, such as conventional plastic materials, are suitable. Various metals, such as stainless steel, are also considered suitable.

The spool 400 can have any suitable dimensions, and a skilled artisan will be able to select suitable dimensions for a particular spool based on various considerations, including the desired final dimensions of the ligating band the spool is intended to be used to form, and the nature of the material from which the ligating band will be formed. The inventor considers it advantageous to size the spool such that the neck portion has an outer diameter that is slightly less than the desired resting diameter of the final ligating band the spool will be used to form.

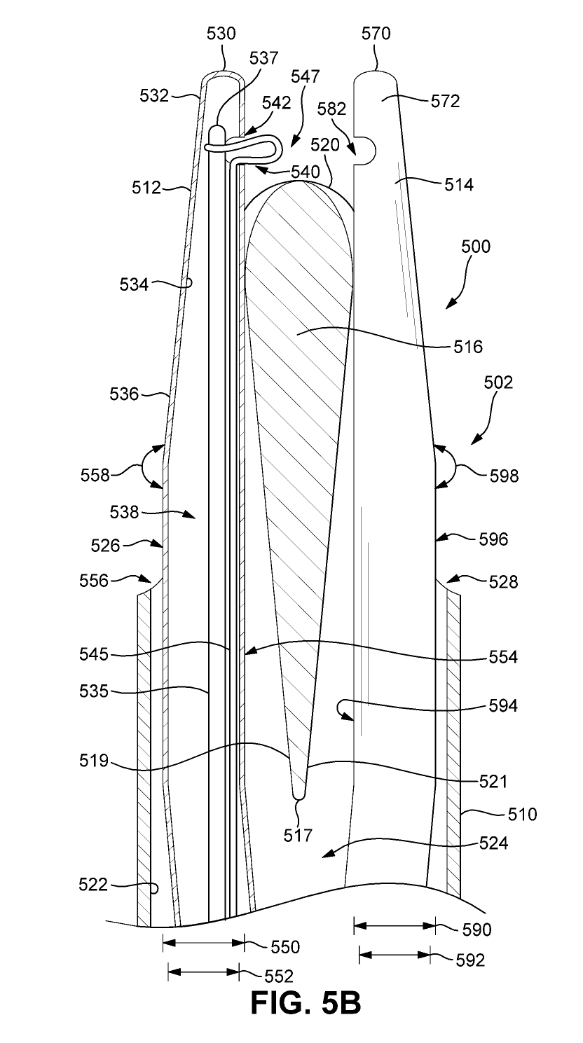

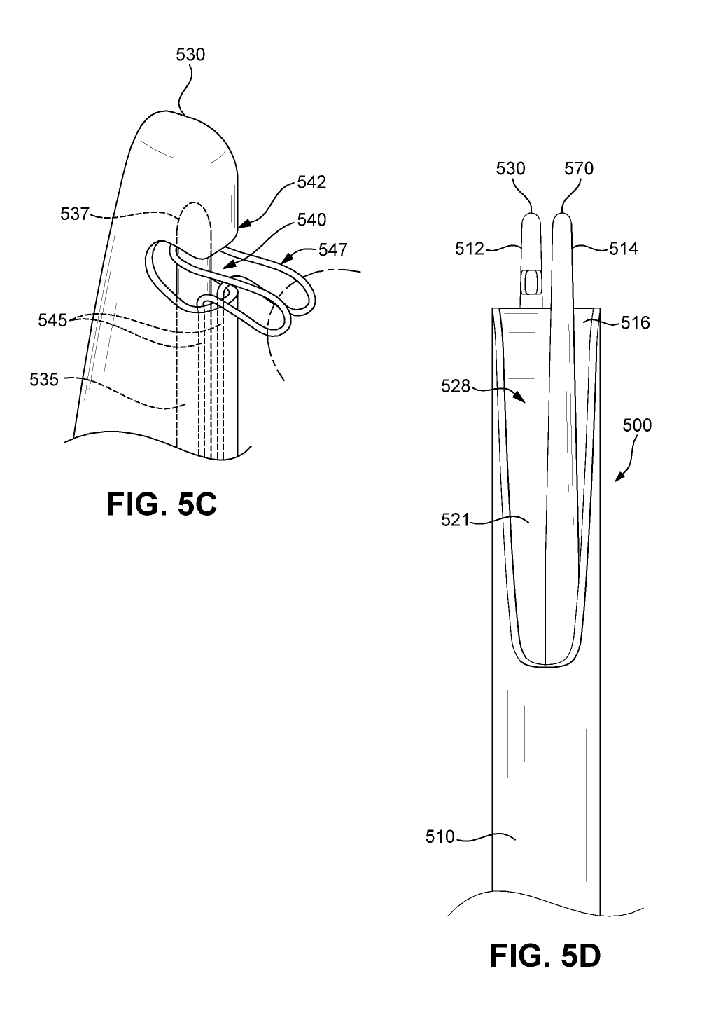

FIGS. 5, 5A, 5B, and 5C illustrate an introducer 500 for holding and transporting a ligating band to a position adjacent a tissue to be ligated. As described below, the introducer 500 can be used for stretching a ligating band to place the ligating band in a stretched configuration, for passing the ligating band over the tissue to be ligated while the ligating band is in its stretched configuration, and for returning the ligating band to its unstretched configuration on the tissue to be ligated so that the ligating band surrounds and constricts the tissue.

It is noted that only the distal end 502 of the introducer 500 is illustrated in the Figures. Skilled artisans will readily understand that the introducer has an opposing proximal end with suitable structure. Furthermore, an introducer and components according to a particular embodiment can have any suitable length, and skilled artisans will be able to select appropriate lengths for such an introducer and its components based on various considerations, including the procedure for which the introducer is intended to be used and the location within the body of the tissue with which the introducer is intended to be used.

The introducer 500 comprises an elongate tubular member 510, first 512 and second 514 extendable arms, and a separating bridge 516.

The elongate tubular member 510 has a proximal end (not illustrated in the figures), a distal end 520, and an inner surface 522. The elongate tubular member 510 defines a passageway 524 extending from the proximal end to the distal end 520. The separating bridge 516 is disposed within the passageway 524 near the distal end 520 of the elongate tubular member 510 and extends across the entire passageway 524 from a first portion of the inner surface 522 to an opposing second portion of the inner surface 522 relative to the lengthwise axis of the elongate tubular member 510.

The distal end 520 of the elongate tubular member 510 defines first 526 and second 528 notches that open to the distal end 520. Each of the notches 526, 528 provides access to the passageway 524 and a space from which one of the arms 512, 514 can extend, as described more fully below. Each of the notches 526, 528 can have any suitable shape. The shapes illustrated in the figures, in which each of the notches 526, 528 has a relatively narrow base toward the proximal end of the elongate tubular member 510 and a relatively wide opening at the distal end 520 of the elongate tubular member 510, is considered advantageous at least because the shape provides the desired space for extension of one of the arms 512, 514 without significant compromise to the structural integrity of the distal end 520 of the elongate tubular member 510. Furthermore, additional notches can be provided if additional arms are included in a particular introducer.

The first arm 512 is a hollow body having a proximal end (not illustrated in the figures), a distal end 530, and a wall 532 with an inner surface 534 and an outer surface 536. The wall 532 defines an inner passageway 538. An opening 540 extends through the thickness of the wall 532 near the distal end 530 of the first arm 512 and provides access to the inner passageway 538. The wall 532 advantageously defines a notch 542 that defines the opening 540.

The first arm 512 has a first outer diameter 550 and a second outer diameter 552. The first outer diameter 550 is greater than the second outer diameter 552. The first arm 512 tapers from the first outer diameter 550 to the second outer diameter 552. In the embodiment illustrated in the figures, the first arm 512 has a first side 554 that is substantially linear and a second side 556 that includes an angle 558 that is greater than 180° . The second side 556 in this embodiment defines an inward taper that transitions from the first outer diameter 550 to the second outer diameter 552.

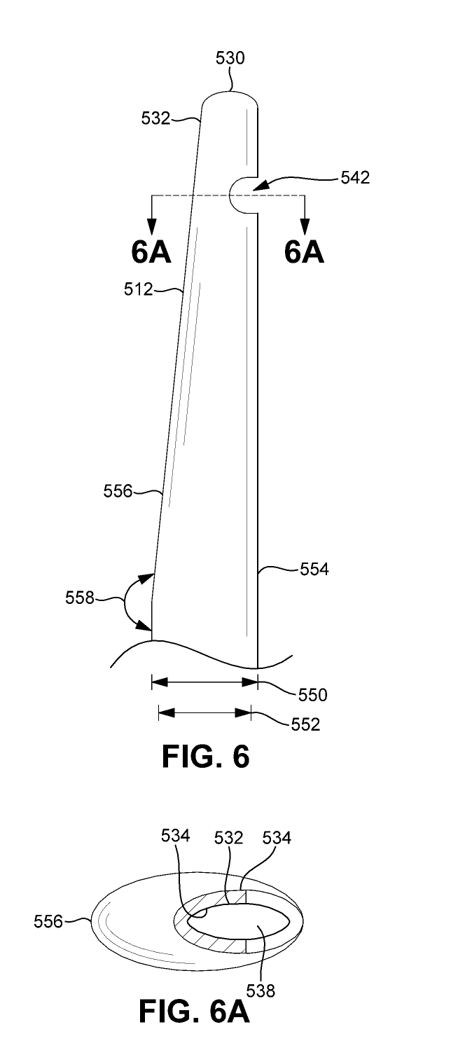

The first arm 512 is illustrated in FIGS. 6 and 6A independent of the introducer 500.

A first inner member 535 is disposed within the inner passageway 538 of the first arm 512. The first inner member 535 extends from the proximal end (not illustrated in the figures) of the first arm 512 and toward the distal end 530 of the first arm 512. As best illustrated in FIG. 5B, the first inner member 535 advantageously extends axially beyond the opening 540 that provides access to the inner passageway 538. In this manner, a first engaging member 545 can be looped around the distal end 537 of the first inner member 535 to form a band engaging loop 547. The ends of the first engaging member 545 extend through the inner passageway 538 of the first arm 512 to or axially beyond the proximal end (not illustrated in the figures) of the first arm 512. This arrangement allows a user to hold or otherwise retain one end of the first engaging member 545 while releasing the other end such that the other end of the first engaging member 545 can be pulled through and eventually released from the first inner member 535 to eliminate the band engaging loop 547, effectively releasing anything held within the band engaging loop 547 (such as a ligating band, as described below).

The first inner member 535 can comprise any suitable structure. Examples of suitable structures include an elongate rod member, such as a metal or plastic rod member. The first engaging member 545 can comprise any suitable structure. Examples of suitable structures include a thread, filament, string, and the like. Medical grade sutures are also considered acceptable, at least because of their ready availability, ease of handling, tensile strength, and well-characterized nature.

The second arm 514 has the same construction as the first arm 512. Thus, the second arm 514 is a hollow body having a proximal end (not illustrated in the figures), a distal end 570, and a wall 572 with an inner surface and an outer surface. The wall 572 defines an inner passageway. An opening extends through the thickness of the wall 572 near the distal end 570 of the first arm 512 and provides access to the inner passageway. The wall 572 advantageously defines a notch 582 that defines the opening 580.

Also, the second arm 514 has a first outer diameter 590 and a second outer diameter 592. The first outer diameter 590 is greater than the second outer diameter 592. The second arm 514 tapers from the first outer diameter 590 to the second outer diameter 592. In the embodiment illustrated in the figures, the second arm 514 has a first side 594 that is substantially linear and a second side 596 that includes an angle 598 that is greater than 180°. The second side 596 in this embodiment defines an inward taper that transitions from the first outer diameter 590 to the second outer diameter 592.

While not illustrated in the figures, the second arm 514 also includes a second inner member, similar to the first inner member, disposed within the inner passageway as described above for the first inner member 535. Similarly, the second arm 514 also includes a second engaging member disposed within the inner passageway as described above for the first engaging member. The second engaging member can be looped around the distal end of the second inner member, as described above for the first engaging member, to form a band engaging loop. The ends of the second engaging member extend through the inner passageway of the second arm 514 to or axially beyond the proximal end (not illustrated in the figures) of the second arm 514. Again similar to the first engaging member 535, this arrangement allows a user to pull one end of the second engaging member such that the other end of the second engaging member eventually releases from the second inner member to eliminate the second band engaging loop, effectively releasing anything held within the second band engaging loop (such as a ligating band, as described below).

As best illustrated in FIG. 5B, the separating bridge 516 is disposed within the passageway 524 near the distal end 520 of the elongate tubular member 510 and extends across the entire passageway 524 from a first portion of the inner surface 522 to an opposing second portion of the inner surface 522 relative to the lengthwise axis of the elongate tubular member 510. The separating bridge 516 can have any suitable configuration, shape and size, and a skilled artisan will be able to select an appropriate configuration, shape and size for a separating bridge in an introducer according to a particular embodiment based on various considerations, including the configuration of the elongate tubular member and the configuration and number of arms in the introducer. Examples of suitable separating bridges include a member having oppositely positioned sides that slope proximally-inward toward the proximal end of the elongate tubular member 510 to a rounded edge that forms the proximal end of the separating bridge 516. The wedge-shaped member illustrated in the figures is considered advantageous at least because it provides a minimal proximal edge 517 in the passageway 524, which avoids engagement of the arms as they are extended through the elongate tubular member during assembly or otherwise, while providing opposing ramped surfaces 519, 521 that deflect the distal ends 530, 570 of the arms 512, 514 outward through the notches 526, 528 in the elongate tubular member 510 as the arms are advanced distally within the passageway 524. Other suitable configurations for the separating bridge 516 include an airfoil configuration, an elliptical configuration, a diamond configuration, a lozenge configuration, and a circular configuration.

The separating bridge 516 can comprise a separate member disposed within the passageway 524 of the elongate tubular member 510 and attached to the inner surface 522 of the elongate tubular member 510, such as by adhesive or another suitable means for attaching. Alternatively, the separating bridge 516 can be integrally formed with the elongate tubular member 510.

Each of the components of the introducer 500 can be formed of any suitable material, and a skilled artisan will be able to select a suitable material for each component of a particular introducer based on various considerations, including the nature of any sterilization procedures to which the introducer will be exposed. Various polymeric materials, such as conventional plastic materials, are considered suitable for all components.

While the arms 512, 514 can be formed of any suitable material, the inventor has determined that the arms 512, 514 are advantageously formed of stainless steel or other suitable metal. Specifically, each of the arms 512, 514 advantageously comprises a hollow tube formed of metal, such as stainless steel. As described below, an outward bias is advantageously placed on each of the arms 512, 514 such that, when a constraining force is removed from the distal ends 530, 570 of the arms 512, 514—such as the force placed on the arms 512, 514 when positioned within the elongate tubular member 510 of the introducer 500—the distal ends of the arms 512, 514 splay outward. Any suitable technique for introducing an outward bias on a body can be used during the formation of the arms for an introducer according to a particular embodiment, and a skilled artisan will be able to select an appropriate technique for particular arms based on various considerations, including the material used to form the arms. The inventor has determined that a curling procedure on arms formed of hollow stainless steel tubes confers a suitable outward bias on the arms.

While stainless steel is currently considered desirable for forming the arms, other suitable materials can be used, including nickel titanium alloys, such as Nitinol, and other suitable shape memory materials, and cold drawn cobalt chromium alloys, such as ASTM F562 and ASTM F 1058 (commercial examples of which include MP35N™ and Elgiloy™, both of which are available from Fort Wayne Metals, Fort Wayne, Ind.; MP35N is a registered trademark of SPS Technologies, Inc. (Jenkintown, Pa., USA); Elgiloy is a registered trademark of Combined Metals of Chicago LLC (Elk Grove Village, Ill., USA)).

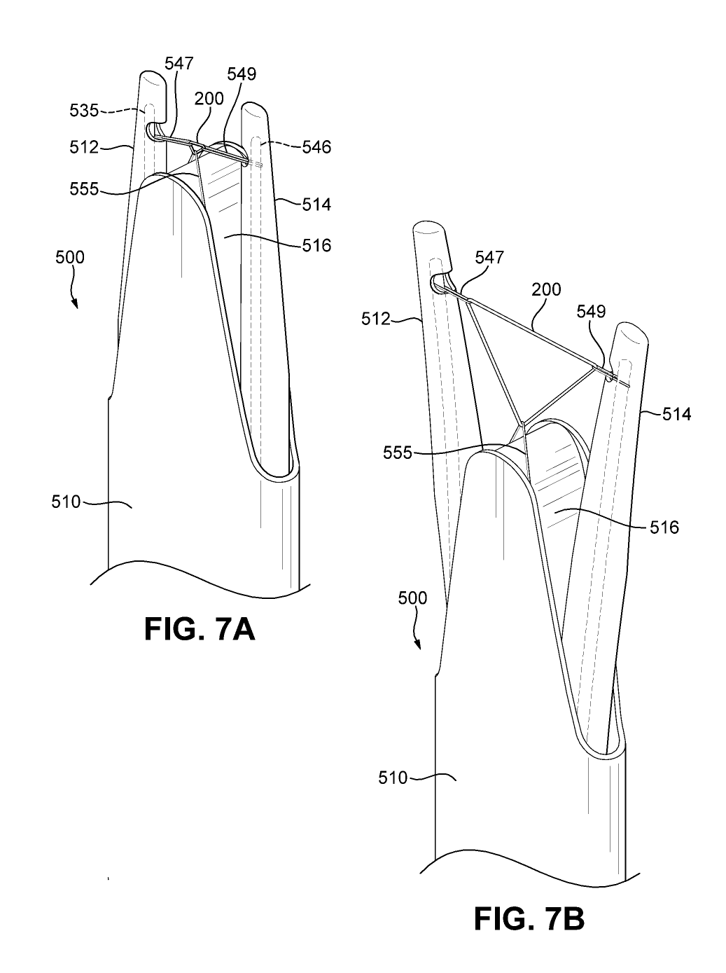

FIGS. 7A and 7B illustrate the introducer 500 with the ligating band 200 held between the arms 512, 514 by the first engaging loop 547 formed by the first engaging member (not illustrated in the figures) and the second engaging loop 549 formed by the second engaging member (not illustrated in the figures). FIG. 7A illustrates the arms 512, 514 of the introducer in a retracted position and the ligating band 200 in an unstretched configuration. FIG. 7B illustrates the arms 512, 514 of the introducer 500 in an extended position and the ligating band 200 in a stretched configuration.

A third engaging member 555 is disposed within the passageway 524 of the elongate tubular member 510. Similar to the engaging members disposed within the arms 512, 514, the third engaging member 555 extends to or beyond the proximal end of the elongate tubular member 510 and loops around the ligating band 200. In this manner, the third engaging member 555 provides another point at which a pulling force can be applied to the ligating band 200 as the arms 512, 514 are extended distally, effectively assisting in the stretching of the ligating band 200. The third engaging member 555 is similar to the first and second engaging members, and can formed of the same or different materials as those members.

In the extended position, each of the arms 512, 514 has been distally advanced to extend further beyond the distal end of the elongate tubular member 510. The separating bridge 516 has helped to splay each of the arms 512, 514 radially outward relative to a longitudinal axis of the elongate tubular member. As a result of this outward movement of the arms 512, 514, the first engaging loop 547 and the second engaging loop 549, each of which is passed around a portion of the ligating band 200, has pulled on the ligating band 200 to stretch the band 200 into the stretched configuration.

In this stretched configuration, the ligating band 200 can be passed over a tissue or portion of a tissue to be ligated, such as an LAA of a patient, by manipulating the entire introducer 500 or one or both of the arms 512, 514 until the desired relative positioning between the ligating band 200 and the tissue or portion of a tissue to be ligated has been achieved. Once the stretched ligating band 200 has been placed over the tissue, one end of the first engaging member 545 and one end of the second engaging member (not illustrated in the figures) can be pulled proximally through the respective inner passageway of the respective arm 512, 514 until the engaging loops 547, 549 are eliminated and the ligating band 200 is released. Similarly, one end of the third engaging member 555 can be pulled proximally to release it from around the ligating band 200. Once all engaging members have been removed from around the ligating band 200, the ligating band 200 will revert to the unstretched configuration around the tissue to be ligated. At this time, the introducer 500 can be retracted and removed from the body. The arms 512, 514 can be retracted into the elongate tubular member 510 before, during or after retraction of the elongate tubular member 510, if desired.

A ligating band can be loaded onto the introducer 500 in any suitable manner, and a skilled artisan will be able to determine an appropriate technique based on various considerations, including the material of the ligating band and the structure of the introducer.

As an example, a ligating band, such as ligating band 200 illustrated in FIG. 2, can be transferred from a holding and/or forming apparatus, such as spool 400 illustrated in FIG. 4, onto the introducer 500 as follows. First, an end of a suture or other suitable engaging member is passed between the ligating band and the holding and/or forming apparatus, such as through one of the channels 480, 482 in spool 400. The same end of the engaging member is then passed back between the ligating band and the spool such that a loop is formed in the engaging member on one side of the ligating band and spool and the free ends of the engaging member extend away from the other side of the ligating band and spool. These steps can be repeated one or more times with another engaging member on another side of the ligating band and spool if more than one engaging member is to be used. For each engaging member used, the free ends are then passed through the inner passageway of one of the arms 512, 514, from the opening to the inner passageway to the proximal end of the arm. The loop formed in the engaging member above is then passed into the opening of the same arm and the inner member disposed within the inner passageway of the same arm is extended distally within the inner passageway of the arm through the loop to secure the engaging member in place. This forms a band engaging loop around the distal end of the ligating member and the ligating band, as described above. This process is repeated for each arm, using a separate engaging member. If included, one end of another engaging member is passed alongside each side of the separating bridge and through the inner passageway of the elongate member. Once all engaging members are in place, they can be temporarily secured. The arms 512, 514 can then be partially extended to induce some stretching in the ligating band, which will allow the spool to be freed. The arms 512, 514 can then be retracted to allow the ligating band to return to the smallest diameter the introducer will allow. At this time, the ligating band is considered ready for placement.

Once loaded with a ligating band, the introducer 500 can be used in a method of ligating tissue, such as method 100 illustrated in FIG. 1 and described above. Example 1 describes an exemplary method of ligating tissue that uses the introducer 500.

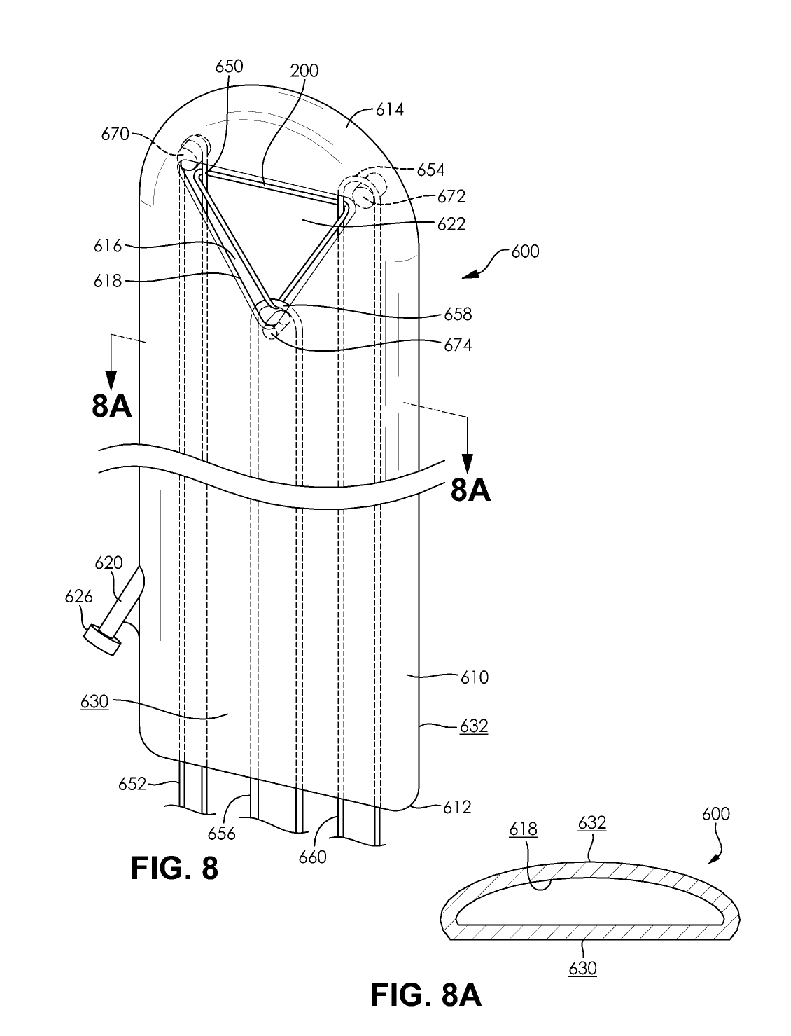

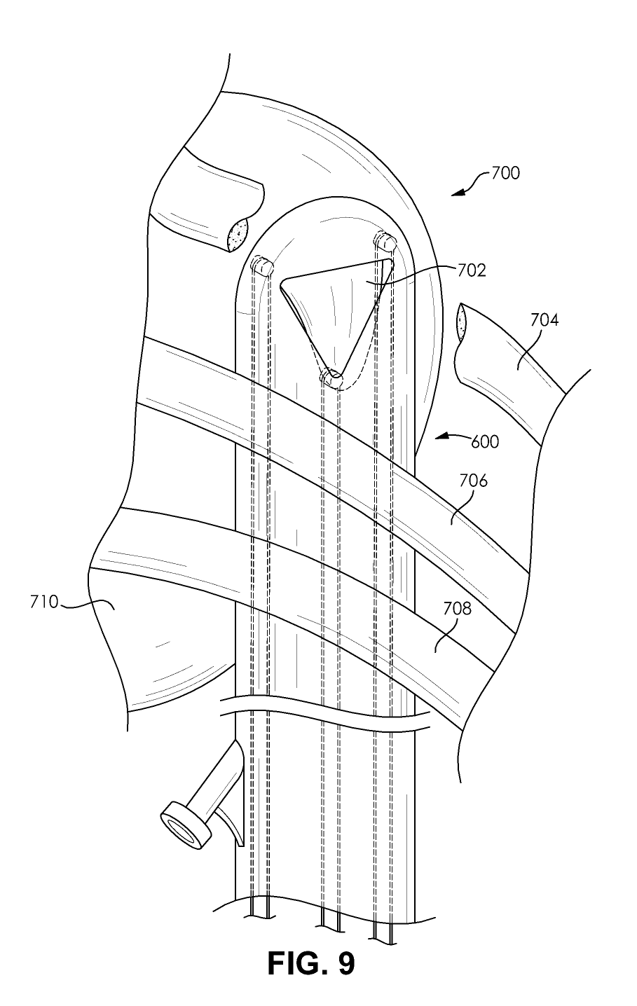

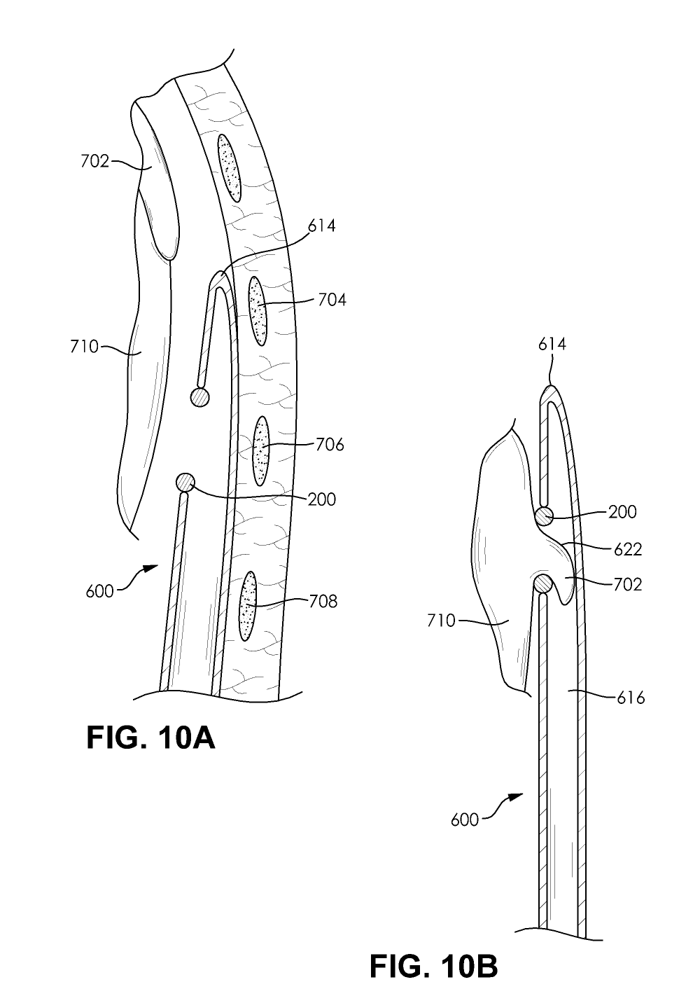

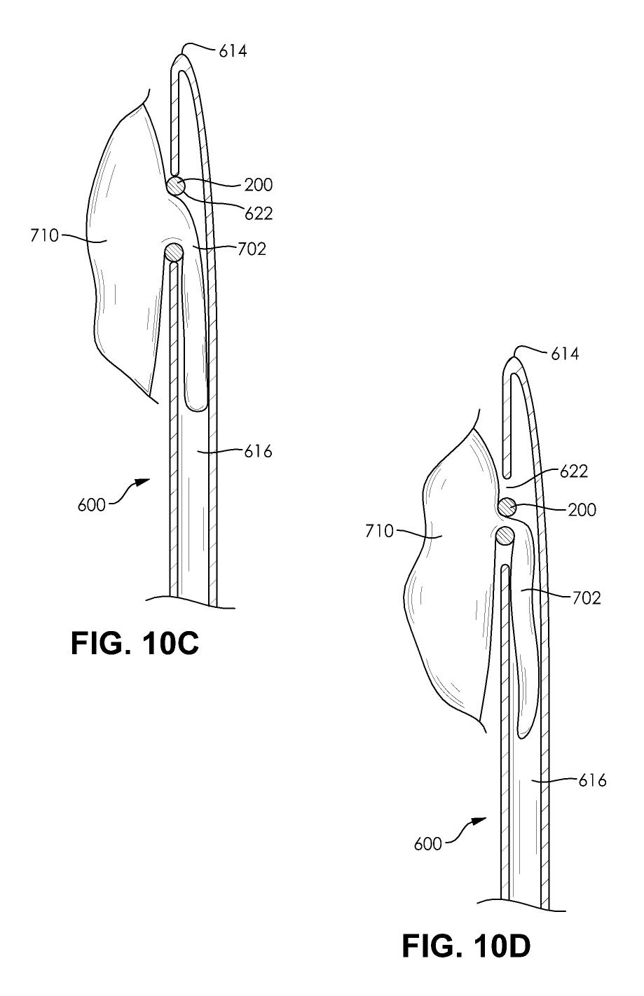

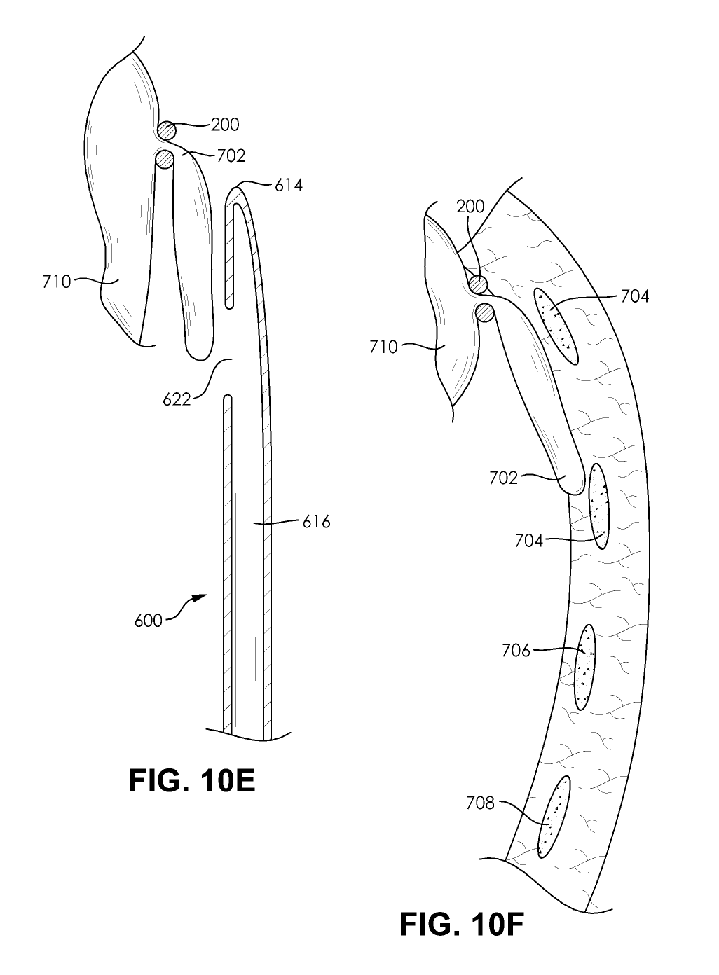

FIGS. 8 and 8A illustrate another introducer 600 suitable for use with the ligating band 200. FIG. 9 illustrates a chest cavity 700 of a patient in which the introducer 600 is being used in a method of ligating an LAA 702. Each of FIGS. 10A, 10B, 10C, 10D, 10E and 10F illustrate the introducer 600 in a position during use in a method of ligating tissue. In this embodiment, the introducer 600 includes a main body 610 that has a closed proximal end 612 and a closed distal end 614 and that defines a passageway 616 bounded by inner wall 618. The proximal end 612 defines a port 620 and the distal end 614 defines an opening 622. The passageway 616 extends between the port 620 and the opening 622. In this embodiment, the introducer 600 is able to maintain the ligating band 200 in a stretched position adjacent the opening 622, such as illustrated in FIG. 8, by a first engaging loop 650 formed by a first engaging member 652, a second engaging loop 654 formed by a second engaging member 656, and a third engaging loop 658 formed by a third engaging member 660. Each of the engaging members 652, 656, 660 has two ends that exit the main body 610 of the introducer, such as through an opening having a seal disposed in it, which can be disposed at, on, adjacent or near the proximal end 612. This configuration allows a user to manipulate each of the engaging members 652, 656, 660 by pulling one end of the respective engaging member 652, 656, 660 and allowing the other end of the respective engaging member 652, 656, 660 to pass through the ligating band 200 to ultimately release the ligating band 200 from the engaging loop 650, 654, 658 formed by the respective engaging member 652, 656, 660.

The port 620 provides fluid access to the passageway 616 from an external environment. As such, the port 620 defines an opening in a first inner wall 630 of the main body 610. As best illustrated in FIGS. 8 and 9, the port 620 can define a sidearm that defines a subpassageway that is in fluid communication with the passageway 616 of the main body 610, and can include any suitable fittings, connectors or other structure adapted for sealing the port 620 or attaching another element to the port in a manner that provides fluid communication between the other element and the passageway 616. For example, in the illustrated embodiment, the port 620 defines a sidearm that defines a subpassageway 624 in fluid communication with the passageway 616 and that includes a luer lock fitting 626 adapted for connection to mating connectors. As described in more detail below, a section of tubing or other element that is connected to a vacuum source can be attached to the port 620 using the fitting 626.

The opening 622 provides fluid access to the passageway 616 from an external environment, and is sized and configured to allow passage of a portion of a target tissue through the opening 622 and into the passageway 616. Accordingly, the opening 622 can have any suitable size and configuration. A skilled artisan will be able to select a suitable size and configuration for an opening in an introducer according to a particular embodiment based on various considerations, including the size, configuration and location of a target tissue for which the introducer is intended to be used to ligation and the size and configuration of a ligating band with which the introducer is intended to be used. In the illustrated embodiment, the opening 622 has a triangular configuration and, as described in more detail below, is sized to allow passage of a human LAA through the opening 622 and into the passageway 616. The triangular shape also allows use of a desirable number of engaging members to maintain the ligating band in a stretched configuration. Other examples of suitable configurations for the opening include circular, ovoid, square, rectangular, and other polygonal shapes.

In the illustrated embodiment, the main body 610 is an elongate, flattened member with opposing first 630 and second 632 outer walls. The first outer wall 630 defines a flat surface and the second outer wall 632 defines a rounded surface that extends from one lateral side of the first outer wall 630 to an opposing second lateral side of the first outer wall 630. The passageway 616 has a substantially constant cross-sectional area along the length of the main body, as best illustrated in FIG. 10B. The distal end 614 of the main body 610, however, defines a taper such that the cross-sectional area of the passageway 616 reduces as the passageway 616 extends toward the distal end 614. This overall structure and configuration provides a relatively low profile introducer 600 that is well suited for use in tissue ligation methods during which access to or passage through relatively narrow spaces is possible or expected. As illustrated in FIGS. 10A through 10F, this makes the introducer 600 particularly well-suited for use in ligation of the LAA.

The engaging members 652, 656, 660 are similar to the engaging members described above in connection with the introducer illustrated in FIGS. 5A through 5D and FIGS. 7A and 7B. Thus, each of the engaging members 652, 656, 660 can comprise any suitable structure. Examples of suitable structures include a thread, filament, string, and the like. Medical grade sutures are also considered acceptable, at least because of their ready availability, ease of handling, tensile strength, and well-characterized nature.

The introducer 600 includes structure that facilitates formation of the first 650, second 654, and third 658 engaging loops by the first 652, second 656, and third 658 engaging members. For example, in the illustrated embodiment, introducer 600 includes 670, 672, and 674. Each of the posts 670, 672, 674 comprises a solid member that extends from a first location on the inner wall 618 to a second location on the inner wall 618, thereby spanning the passageway 616. Each post 670, 672, 674 can be integrally formed with the main body 610 of the introducer 600, or can comprise separate members that are attached to the main body 610, such as in the positions illustrated in FIG. 8.

The introducer 600 can include other structure that facilitates formation of the first 650, second 654, and third 658 engaging loops by the first 652, second 656, and third 658 engaging members. For examples, as an alternative to or in addition to the posts 670, 672, 674 described above, the first outer wall 630 can define a channel for each engaging member 652, 656, 658. Each of the engaging members 652, 656, 658 can be passed through its respective channel and through the ligating band 200.

FIG. 9 illustrates the introducer 600 positioned in a chest cavity 700 of a patient, with the LAA 702 of the patient passed through the opening 622 and into the passageway 616 of the main body 610. The introducer 600 has been passed under a series of ribs 704, 706, 708 and positioned adjacent the heart 710. The low profile nature of the introducer 600 makes it particularly well-suited for use in ligation of tissue accessible through narrow passages such as illustrated with the LAA 702.

Inclusion of the port 620 allows the introducer 600 to be used in methods of ligating tissue that include a step of applying vacuum pressure to a tissue being ligated. For example, the introducer 600 can be used in the method 100 described above and illustrated in FIG. 1. Alternatively, the introducer can be used in a method of ligating tissue that is similar to method 100, with the following changes. First, in the step 106 of passing the ligating band over the tissue, the ligating band is passed over the tissue by passing the tissue through the opening 622 of the main body and into the passageway 616.

Also, when the introducer 600 is used in a method of ligating tissue, an alternative step of applying vacuum pressure to a tissue can be included. This step, if included, can be accomplished by forming a connection between a vacuum source and the port 620, such as by connecting a suitable tubing line extending from a vacuum source to a fitting on the port 620, and by activating the vacuum source. The vacuum source can be activated in any suitable manner, such as by activating a vacuum pump that provides the vacuum source, by opening a subpassageway define by the port 620, or any other manner suitable for the vacuum source and introducer being used.

If included, the step of applying vacuum pressure to a tissue can be performed before, during, and/or after the step 106 of passing the ligating band over the tissue. Performing this step while the step 106 of passing the ligating band over the tissue is being performed can help to place the ligating band at the base of the tissue to be ligated by maximizing a free portion of the tissue that is disposed in the passageway 616 of the introducer 600 prior to the step of returning the ligating band to an unstretched configuration 110.

### EXAMPLE I

### Method of Ligating Tissue Using a Ligating Band and Introducer

In this exemple method, an extendable arm introducer, such as the introducer 500 illustrated in FIGS. 5, 5A, 5B, and 5C, and described above, is used to place a ligating band, such as ligating band 200 illustrated in FIG. 2 and described above, around a tissue appendage to be ligated.

1. The introducer loaded with a ligating band is introduced into the body via a body lumen, vessel, or created passageway in order to access the appendage requiring ligation.

2. The introducer is manipulated into a position whereas its long axis is perpendicular to the long axis of said appendage.

3. The introducer is actuated such that the arms are advanced distally relative to the distal end of the elongate member. This causes the distal ends of the arms to splay radially through the notches of the distal end of the elongate member relative to the lengthwise axis of the elongate member. As a result, the ligation band stretches open a minimal amount that will accommodate the passage therethrough of said appendage.

4. An additional device may be introduced into the common passageway that has the ability to manipulate the appendage an effective distance into the opening of the expanded ligation band, and-or secure it in said position.

5. The introducer may or may not be “un-actuated” to allow the ligation band to return to a smaller diameter.

6. Once the user is satisfied with placement of the ligation band around the “neck” of the appendage, the proximal end of the inner members disposed within the inner passageways of the arms of the introducer may be moved proximally, thus releasing the engaging members secured by the distal ends of said inner members. The first and second engaging members may be removed proximally through the introducer if desired.

7. A first proximal end of the third engaging member may be released and the second proximal end removed proximally, thus releasing the ligation band from its securement to the separating bridge.

8. The introducer may be completely “un-actuated” to lower its profile and prepare it for removal from the body.

9. The result may be inspected via endoscope, and the process repeated if deemed necessary by the user.

10. The ligating band should cause necrosis of the appendage followed by scarring-over of the appendage base, and eventual bio-absorption of the appendage and ligation band.

### EXAMPLE II

### Method of Ligating Tissue Using a Ligating Band and Introducer

FIGS. 10A through 10F illustrate an example method of ligating tissue in which the introducer 600 illustrated in FIGS. 8, 8A and 9 is used to ligate the LAA 702 of a patient using ligating band 200.

In FIG. 10A, the distal end 614 of the introducer 600 is being passed under ribs 704, 706, 708 and positioned adjacent the heart 710. The ligating band 200 is positioned adjacent the opening 622 and in a stretched configuration.

In FIG. 10B, the distal end 614 of the introducer 600 has been maneuvered such that a portion of the LAA 702 has passed through the opening 622 and into the passageway 616 defined by the main body 610. As a result, the ligating band 200 has been passed over the LAA 702.

In FIG. 10C, vacuum pressure has been applied to the passageway 616 of the introducer 600, such as by forming a connection between the passageway and an external vacuum source via the port (not illustrated in the Figure) of the introducer 600, as described above. As a result, a larger portion of the LAA 702 has been drawn through the opening 622 and into the passageway 616, and the opening 622 has been positioned around the base of the LAA 702 near its interface with the heart 710.

In FIG. 10D, the ligation band 702 has been released from the engaging loops formed by the engaging members (not illustrated in the Figure) to return the ligating band 200 to an unstretched configuration. As a result, the ligating band 200 is placed around the base of the LAA 702 near its interface with the heart 700. As a result, the base of the LAA 702 has been constricted.

In FIG. 10E, the vacuum pressure has been released and the introducer 600 has been withdrawn such that the LAA 702 has exited the passageway 616 by passing through the opening 622. At this point, the LAA 702, with the ligating band 200 disposed around its base, is left adjacent the heart 710.

In FIG. 10F, the introducer 600 has been removed from the chest cavity 700, leaving the ligating band 200 around the base of the LAA 702. Eventually, the constriction provided by the ligating band 200 will produce necrosis of the LAA 702 and eventual sloughing off from the heart 710 and bio-absorption of the LAA 702 and ligation band 200.

The foregoing disclosure includes the best mode of the inventor for practicing the invention. It is apparent, however, that those skilled in the relevant art will recognize variations of the invention that are not described herein. While the invention is defined by the appended claims, the invention is not limited to the literal meaning of the claims, but also includes these variations.

What is claimed is:

1. A method of ligating tissue in a body of a patient, comprising:
placing a ligating band on an introducer, the ligating band having an unstretched configuration and a stretched configuration and comprising an elongate fiber having first and second ends and forming a series of loops;
stretching the ligating band to place the ligating band in the stretched configuration;
transporting the ligating band in the stretched configuration to a position adjacent said tissue by moving the introducer within said body of said patient;
passing the ligating band over said tissue while the ligating band is in the stretched configuration; and
releasing the ligating band from the introducer to place the ligating band in the unstretched configuration around said tissue.

2. The method of claim 1, wherein the ligating band comprising a natural material.

3. The method of claim 2, wherein the ligating band comprises a natural silk.

4. The method of claim 3, wherein the ligating band comprises spider silk.

5. The method of claim 4, wherein the ligating band comprises spider silk that has been treated to remove or reduce an outer sticky layer.

6. The method of claim 3, wherein the ligating band comprises silkworm silk.

7. The method of claim 2, wherein the ligating band comprises a recombinantly produced form of a natural silk.

8. The method of claim 2, wherein the ligating band comprises a bioremodelable material.

9. The method of claim 8, wherein the ligating band comprises an extracellular matrix material.

10. The method of claim 9, wherein the ligating band comprises small intestine submucosa.

11. The method of claim 1, wherein the first and second ends of the elongate fiber are not directly secured to each other.

12. The method of claim 11, wherein the second end is secured to a portion of the elongate fiber positioned within a loop of the series of loops.

13. The method of claim 12, wherein the second end is adhered to the portion of the elongate fiber positioned within a loop of the series of loops.

14. The method of claim 12, wherein the second end has been felted to the portion of the elongate fiber positioned within a loop of the series of loops.

15. The method of claim 1, wherein the introducer defines a passageway and an opening to the passageway; and
wherein the step of passing the ligating band over said tissue comprises passing said tissue through the opening and into the passageway.

16. The method of claim 15, further comprising applying vacuum pressure to said tissue.

17. The method of claim 15, wherein the applying vacuum pressure to said tissue is performed during the step of passing the ligating band over said tissue to facilitate the passing of said tissue through the opening and into the passageway.

18. A method of ligating tissue in a body of a patient, comprising:
placing a ligating band on an introducer defining a passageway and an opening to the passageway, the ligating band having an unstretched configuration and a stretched configuration and comprising an elongate fiber having first and second ends and forming a series of loops;
stretching the ligating band to place the ligating band in the stretched configuration;
transporting the ligating band in the stretched configuration to a position adjacent said tissue by moving the introducer within said body of said patient;
passing the ligating band over said tissue while the ligating band is in the stretched configuration;
applying vacuum pressure to said tissue through the passageway while the step of passing the ligating band over said tissue is being performed to facilitate passing said tissue through the opening and into the passageway; and
releasing the ligating band from the introducer to place the ligating band in the unstretched configuration around said tissue.

19. The method of claim 18, wherein the ligating band comprises a natural silk.

20. A ligating band for ligating tissue, comprising:
an elongate fiber of natural silk having first and second ends and forming a series of loops;
wherein the first and second ends of the elongate fiber are not directly secured to each other; and
wherein the second end is secured to a portion of the elongate fiber positioned within a loop of the series of loops.
